(19) United States
(12) Patent Application Publication
(10) Pub. No.: US 2005/0240215 A1
(43) Pub. Date: Oct. 27, 2005

(54) Magnetic embolic protection device and method

(75) Inventor: Louis G. Ellis, St. Anthony, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/828,721

(22) Filed: Apr. 21, 2004

(51) Int. Cl. A61M 29/00

(52) U.S. Cl. 606/200

(57) ABSTRACT

An embolic protection device including an elongate shaft having a proximal end and a distal end. A magnetically permeable section can be disposed proximate the proximal end of the shaft and an embolic protection filter can be disposed on the elongate shaft. And a captivation tool including a magnetic section can be magnetically couplable to the magnetically permeable section disposed on the shaft.

### FIELD OF THE INVENTION

The present invention relates generally to the field of embolic protection. More specifically, the present invention pertains to the apparatus and methods for the placement of embolic protection devices and associated medical devices.

### BACKGROUND OF THE INVENTION

Intravascular devices such as embolic protection filters are generally placed within the lumen of a blood vessel, Saphenous vein graph (SVG) or artery to filter embolic debris dislodged during a therapeutic procedure such as percutaneous transluminal coronary angioplasty (PTCA), percutaneous extraction atherectomy, or stent delivery. To filter this dislodged embolic debris, an embolic protection filter can be placed distally of the therapeutic device (e.g. an angioplasty or atherectomy catheter) and deployed within the patient's vessel or artery. Often it will be necessary to place an embolic protection filter with one catheter, perform angioplasty or atherectomy with another catheter and place a stent all during one session.

### SUMMARY OF THE INVENTION

The present invention relates to the placement of the embolic protection filters. A magnetic coupling can be used to hold an elongate shaft in position while advancing and withdrawing devices over the shaft. Such a device may assist in filter or catheter exchange.

### BRIEF DESCRIPTION OF THE DRAWINGS

FIG. 1 is a view of an emoblic protection filter disposed on an elongate shaft within a vessel, wherein the elongate shaft is magnetically coupled to a captivation tool; and

FIG. 2 is a cross section of the captivation tool of FIG. 1.

### DETAIL DESCRIPTION OF THE INVENTION

The following description should be read with reference to the drawings, in which like elements in different drawings are numbered in like fashion. The drawings, which are not necessarily to scale, depict selected embodiments that are not intended to limit the scope of the invention. Although examples of construction, dimensions, materials and manufacturing processes may be illustrated for the various elements, those skilled in the art will recognize that many of the examples provided have suitable alternatives that may be utilized.

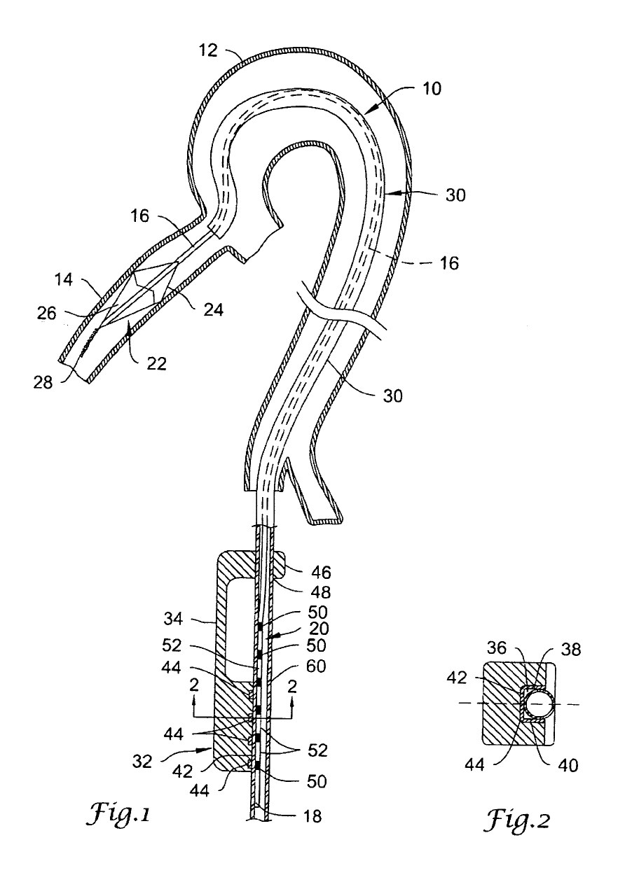

FIG. 1 is a view of an embolic protection device 10 disposed in an aorta 12 and a coronary artery 14 of a patient or SVG. The device includes elongate shaft 16 having a proximal end 18 including an operative segment 20. An embolic protection filter 22 is coupled to shaft 16. Filter 22 can include a frame 24 and a permeable membrane 26 disposed thereon. A spring tip 28 can be attached to shaft 16. Device 10 is shown such that shaft 16 is disposed at least in part within a sheath 30. Sheath 30 can be a retrieval sheath, delivery sheath or a therapeutic catheter, such as an angioplasty catheter or the like.

In use, for example, filter 22 can be positioned distally of a coronary artery lesion. An angioplasty balloon can be advanced over elongate shaft 16 to perform angioplasty on the lesion. The angioplasty catheter can be withdrawn. Then a stent delivery catheter can be advanced to the previously dilated lesion to place a stent.

FIG. 1 also shows a captivation tool 32 which can be used to aid in catheter exchanges over elongate shaft 16. Captivation tool 32 includes a housing member 34. Housing member 34 includes a longitudinal slot 36 defined by a pair of side surfaces 38 and 40 and a bottom surface 42. Slot 36 provides a space with sufficient size with slidably receive sheath 30, or for example, an angioplasty catheter, atherectomy catheter or a stent delivery catheter. The size of slot 36 allows such catheter to longitudinally pass freely through slot 36, yet still restrict lateral movement of such catheter between surfaces 38, 40 and 42. Housing member 32 includes a plurality of magnetic sections 44 disposed about slot 36. Housing member 34 of captivation tool 32 may include a catheter guide 46 having a guide opening 48 disposed therethrough for receipt of a catheter such as sheath 30.

Housing 34 can be made from a substantially non-magnetically permeable material, such as a polymer. Magnetic sections 44 can be made from a strong magnetic material with a large cohesive force (such as neodymium boron iron). Operative segment 20 includes a plurality of magnetically permeable sections 50 secured thereto. Examples of suitable metallically permeable materials are Rodar, manufactured by T.N. Wilbur B. Driver Company and available in tube form from Uniform Tubes of Collegeville, Pa.; Hiperco Alloy 50 manufactured by Carpenter Steel, Reading, Pa.; Permendur or 2V Permendur, listed as high permeable magnetic materials having large saturation flux density in the CRC Handbook of Chemistry and Physics, 47^{th} ed.; or any other material with a suitably large residual induction. Magnetically permeable sections 50 are spaced by a non-magnetically permeable spacers 52.

The size and spacing of the magnetic sections 44 and the size and spacing of magnetically permeable sections 50 are chosen to enhance the longitudinal attractive force between shaft 16 and captivation tool 32. The net force for maintaining the position of shaft 16 relative to tool 32 is governed by the equation Fₙₑₜ=F_{L}−μF_{R} where Fₙₑₜ is the net force available to maintain the position of shaft 16, F_{L} is the longitudinal force of attraction between the tool 32 and shaft 16, F_{R} is the radial force of attraction betweens tool 32 and shaft 16, and μ is the friction coefficient between shaft 16 and sheath 30. Thus to obtain high performance from the device, it is helpful to maximize the force F_{L} and minimize the force F_{R} and the friction coefficient μ. The friction coefficient μ may be reduced through the use of lubricous coatings and materials, and the attractive forces F_{L} and F_{R} may be increased through the use of mathematical modeling techniques known in the art.

In use embolic protection device 10 can be advanced to a target site in a vessel lumen by advancing shaft 16. Sheath 30 may be advanced simultaneously therewith to compress filter 22. Alternately, shaft 16 can be placed and filter 22 and sheath 30 advanced there over, as in the case of a floating filter. Operative section 20 can be magnetically coupled to captivation tool 32 such that sheath 16 or other catheters can be placed or removed while shaft 16 remains in place and the vessel lumen. For example, after sheath 30 is withdrawn, another catheter such as an angioplasty catheter or stent delivery catheter may be advanced over shaft 16 while shaft 16 is held in place by captivation tool 32. Then, at the target site, the therapeutic procedure can be performed. After the procedure is performed, another catheter such as a retrieval device may be advanced over shaft 16 to retrieve filter 22. Although sheath 30 is shown as a delivery catheter in the figures, it should be appreciated that sheath 30 is schematically representative of a therapeutic catheter, retrieval sheath or the like.

Having thus described several embodiments of the present invention, those skilled in the art will readily appreciate that other embodiments may be made and used which fall within the scope of the claims attached hereto. Numerous advantages of the invention covered by this document have been set forth in the forth going description. It will be understand that this disclosure is, in many respects, only illustrative. Changes may be made in details, particularly in matters of shape, size and arrangement of parts without exceeding the scope of the invention.

1. An embolic protection device, comprising:
an elongate shaft having a proximal end and a distal end;
a magnetically permeable section disposed proximate the proximal end of the shaft; and
an embolic protection filter disposed on the elongate shaft.

2. A device in accordance with claim 1, further comprising of plurality spaced apart magnetically permeable sections disposed proximate the proximal end of the shaft.

3. A device in accordance with claim 2, further comprising of plurality of non-magnetically permeable spacers disposed between the magnetically permeable sections.

4. A device in accordance with claim 2, further comprising a captivation tool including a plurality of spaced apart magnetic sections magnetically couplable to the magnetically permeable sections.

5. A device in accordance with claim 1, further comprising a captivation tool including a magnetic section magnetically couplable to the magnetically permeable section.

6. A device in accordance with claim 5, further comprising a sheath being disposed between the magnetically permeable section and the magnetic section.

7. A device in accordance with claim 1, further comprising a delivery sheath disposed at least in part about the shaft.

8. A device in accordance with claim 1, wherein the shaft comprises a wire.

9. A device in accordance with claim 8, wherein the shaft comprises a NiTi alloy.

10. A device in accordance with claim 1, wherein the filter includes a frame including nickel titanium alloy.

11. A device in accordance with claim 1, wherein the filter is fixed to the elongate shaft.

12. The method of placing an embolic protection device in a vessel, comprising:
providing an elongate shaft having a proximal end and a distal end, an embolic protection filter disposed on the shaft and a magnetically permeable section disposed on the shaft;
providing a captivation tool including a magnetic section;
advancing the elongate shaft to a target site in the vessel; and
magnetically coupling the magnetically permeable section to the magnetic section.

13. A method in accordance with claim 12, further comprising advancing the shaft and the filter to the target site simultaneously.

14. A method in accordance with claim 12, further comprising providing a plurality of spaced apart magnetically permeable sections disposed proximate the proximal end of the shaft.

15. A method in accordance with claim 14, further comprising disposing a plurality of non-magnetically permeable spacers between the magnetically permeable sections.

16. A method in accordance with claim 14, further comprising providing the captivation tool with a plurality of spaced apart magnetic sections magnetically couplable to the magnetically permeable sections.

17. A method in accordance with claim 12, further comprising providing the captivation tool with a magnetic section magnetically couplable to the magnetically permeable section.

18. A method in accordance with claim 12, further comprising disposing a sheath between the magnetically permeable section and the magnetic section.

19. A method in accordance with claim 12, further comprising disposing a delivery sheath at least in part about the shaft.

20. A method in accordance with claim 12, wherein the shaft comprises a wire.

21. A method in accordance with claim 20, wherein the shaft comprises a NiTi alloy.

22. A method in accordance with claim 12, wherein the filter includes a frame including a nickel titanium alloy.

23. A method in accordance with claim 12, further comprising fixing the filter to the elongate shaft.

24. A method in accordance with claim 12, further comprising the step of advancing a therapeutic catheter along the elongate shaft to the target site.

25. A method in accordance with claim 24, further comprising withdrawing the therapeutic catheter from the elongate shaft and advancing a retrieval sheath over the shaft to retrieve the filter.

26. A method in accordance with claim 25, further comprising withdrawing the elongate shaft and retrieval sheath from the vessel.
